(19) United States
(12) Patent Application Publication
(10) Pub. No.: US 2003/0212456 A1
(43) Pub. Date: Nov. 13, 2003

(54) Implant for tissue repair

(76) Inventors: John Lipchitz, Watertown, MA (US); Nick Cotton, Westborough, MA (US); Rod Berube, North Attleboro, MA (US)

Correspondence Address: JOEL PETROW, Chief Patent Counsel, SMITH & NEPHEW NORTH AMERICA, 1450 Brooks Road, Memphis, TN, 38116, US

(21) Appl. No.: 10/140,830

(22) Filed: May 9, 2002

(51) Int. Cl. A61F 2/08; A61F 2/02

(52) U.S. Cl. 623/13.17; 623/14.12; 623/15.12; 623/23.72

(57) ABSTRACT

An implant for tissue repair includes a support member, a flexible member coupled to the support member, and a substrate coupled to the support member. The substrate includes a material capable of being seeded with and supporting the proliferation of cells. The support member is capable of distributing a load throughout the flexible member. The flexible member includes parallel elongate elements and the support member serves to maintain a spacing between the parallel elongate elements.

### CROSS REFERENCE TO RELATED APPLICATIONS

This application relates to International Application No. PCT/GB00/04166, filed Oct. 30, 2000, which is incorporated by reference herein in its entirety.

### TECHNICAL FIELD

This application relates to an implant for tissue repair.

### BACKGROUND

Implants can be used for the partial or total replacement and/or repair of damaged tissue, particularly connective tissue, such as ligament, cartilage, bone, meniscus, tendon, and skin. Damage to such tissue is a frequent occurrence, particularly as a consequence of exercise or contact sports. Various approaches have been used to restore the function of damaged tissue. For example, one approach is to suture together the torn ends of a damaged ligament or tendon. Another approach includes replacing the tissue with a permanent prosthesis. A still further approach includes implanting a temporary prosthesis to stabilize the tissue and provide a matrix for healing of the tissue while gradually being absorbed into the body.

### SUMMARY

In one general aspect of the invention, an implant for tissue repair includes a support member, a flexible member coupled to the support member, and a substrate coupled to the support member. The substrate is made of a material capable of being seeded with and/or supporting the attachment and proliferation of cells. The support member is capable of distributing a load uniformly throughout the flexible member.

Implementations may include one or more of the following features. For example, the substrate is attached to the support member. At least a portion of the flexible member is threaded into the support member. At least a portion of the flexible member is molded into the support member.

The substrate is planar in form. The flexible member includes parallel elongate elements. The support member serves to maintain a spacing between the parallel elongate elements. The parallel elongate elements include a braided material.

In one implementation, the support member includes a trough formed on a surface of the support member that impinges the substrate. In another implementation, the support member includes a spike configured to facilitate attachment of the implant to tissue.

The flexible member includes one or more of a woven, knitted, braided, crocheted, straight, or twisted material, and a mixture of these. Thus, the flexible member can include a twisted fiber core and a braided outer core (which is typical of a suture design). The support member includes one or more of a woven, non-woven, knitted, braided, crocheted, straight, or twisted material, and a mixture of these. The support member can include biologically derived materials such as, for example, collagen, tissue engineered collagen, or recombinant collagen. Moreover, the flexible member and/or support member can include viable cells, that is, cells able to develop under favorable conditions.

Additionally, the substrate includes one or more of a woven or non-woven material; a foam; a sponge; a dendritic material; a knitted, braided, a crocheted material; and a mixture of these. In one preferred implementation, the flexible member is made of poly(lactic acid); the support member is made of poly(lactic acid); and the substrate is made of poly(glycolic acid).

One or more of the support member, the flexible member, and the substrate is made of bioresorbable material such as bioresorbable polymers or copolymers comprising hydroxy acids, glycolic acid; caprolactone; hydroxybutyrate; dioxanone; orthoesters; orthocarbonates; or aminocarbonates; polylactic acid, polyglycolic acid, or a mixture of these.

One or more of the support member, the flexible member, and the substrate is made of non-bioresorbable material such as polyesters; polyamides; polyalkenes; poly(vinyl fluoride); polytetrafluoroethylene; carbon fibers; natural or synthetic silk; and mixtures of these materials; or a polyester selected from polyethylene terephthalate and polybutylene terephthalate.

In another implementation, the implant includes cells seeded in the substrate. The substrate includes a carrier medium for holding the cells. In this way, the implant includes biological tissue grown from the cells. The substrate provides an environment conducive to cell attachment and proliferation. Once the implant is implanted, the cells lay down a matrix within which the tissue may repair and form. In another implementation, the substrate is seeded with cells and then placed within a bioreactive product suitable for the cells to lay down a matrix and for forming a tissue.

In another general aspect of the invention, a method of making an implant includes coupling a flexible member to a support member to form a flexible structure such that the support member is capable of distributing a load throughout the flexible member. The method also includes coupling a substrate to the flexible structure, incorporating cells into the substrate, and growing biological tissue within at least a portion of the substrate. The substrate includes a material capable of being seeded with and supporting the proliferation of cells.

In one implementation, the implant is cut into a shape suitable for the selected site.

In a further general aspect of the invention, a method of treating a tissue harvest site include providing an implant and implanting the implant at the harvest site. The implant includes a support member, a flexible member coupled to the support member, and a substrate coupled to the support member and comprising a material capable of being seeded with and supporting the proliferation of cells.

In various implementations, the harvest site includes a rotator cuff, an anterior cruciate ligament, a posterior cruciate ligament, an Achilles tendon, a patellar tendon, a medial collateral ligament, a lateral collateral ligament, a ligament or a tendon in the hand, or a ligament or tendon in the elbow.

Aspects of the implant and method can include one or more of the following advantages. The implants and methods provide an implant with increased structural integrity due to the coupling of the flexible member to the support members. Additionally, the implants and methods provide an implant in which loads are distributed from the tissue evenly throughout the flexible member because the spacing between elongate elements of the flexible member is maintained by the support members. Moreover, cells are able to proliferate throughout the support members and/or the flexible members to generate tissue growth and/or facilitate repair because the substrate spans between support members and overlays a substantial portion of the flexible member.

Other features and advantages will be apparent from the description, the drawings, and the claims.

### DESCRIPTION OF DRAWINGS

FIGS. 1 and 2 are perspective views of a first implementation of an implant.

FIG. 3 is a cross-sectional view of the implant of FIGS. 1 and 2 taken along lines 3-3 in FIG. 2.

FIGS. 4-7 are perspective views of a flexible member, a support member, and a substrate, respectively, in the implant of FIGS. 1 and 2.

FIG. 7 is a perspective view of the implant being attached to tissue.

FIG. 8 is a perspective view of another implementation of the flexible member of the implant of FIGS. 1 and 2.

FIG. 9 is a perspective view of a second implementation of the implant.

FIG. 10 is a perspective view of a first implementation of a support member of the implant of FIG. 9.

FIG. 11 is a perspective view of a second implementation of a support member of the implant of FIG. 9.

FIG. 12 is a cross-sectional view of the implant of FIG. 9 taken along lines 12-12.

FIG. 13 is a cross-sectional view of the implant of FIG. 9 taken along lines 13-13.

FIG. 14 is a perspective view of a another implementation of the flexible member of the implant of FIGS. 1, 2, and 8.

FIG. 15 is a perspective view of a third implementation of the implant.

FIG. 16 is a cross-sectional view of the implant of FIG. 15 showing a first implementation of a support member.

FIG. 17A is a cross-sectional view of the implant of FIG. 15 showing a second implementation of a support member.

FIG. 17B is a cross-sectional view of the support member of FIG. 17A.

FIG. 18 is a perspective view of a fourth implementation of the implant.

Like reference symbols in the various drawings indicate like elements.

### DETAILED DESCRIPTION

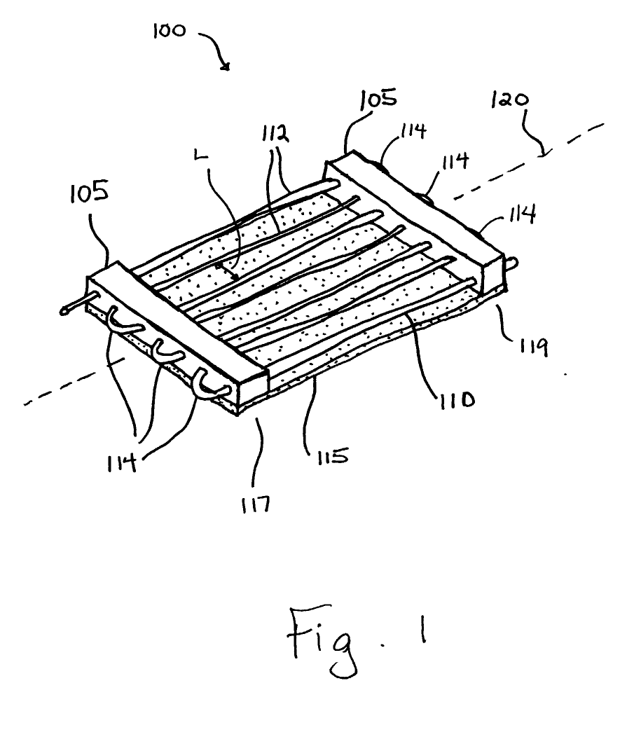

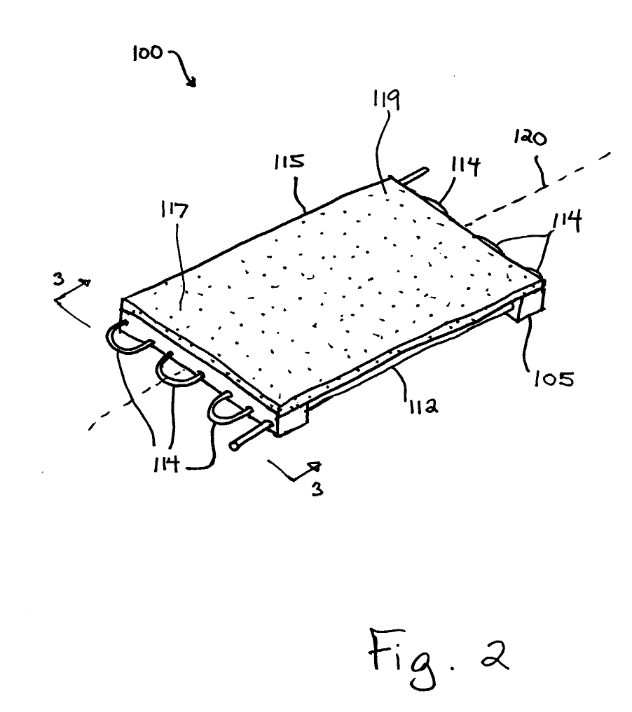

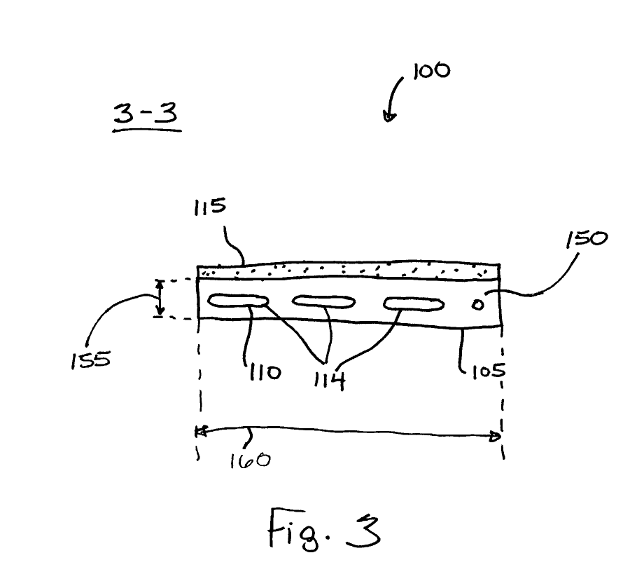

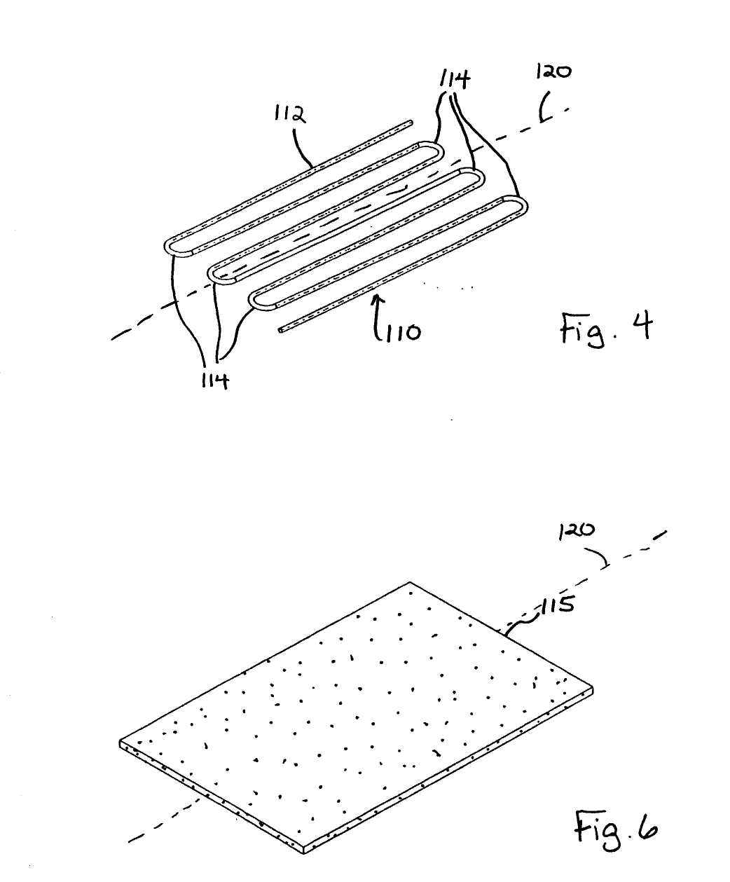

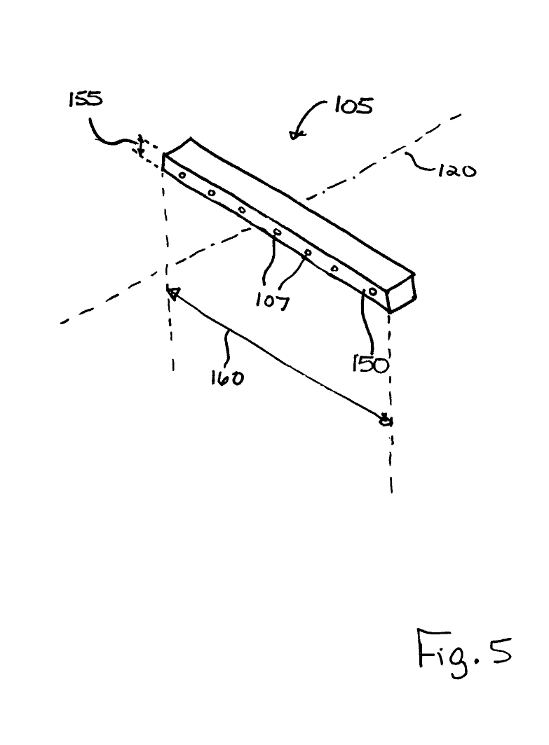

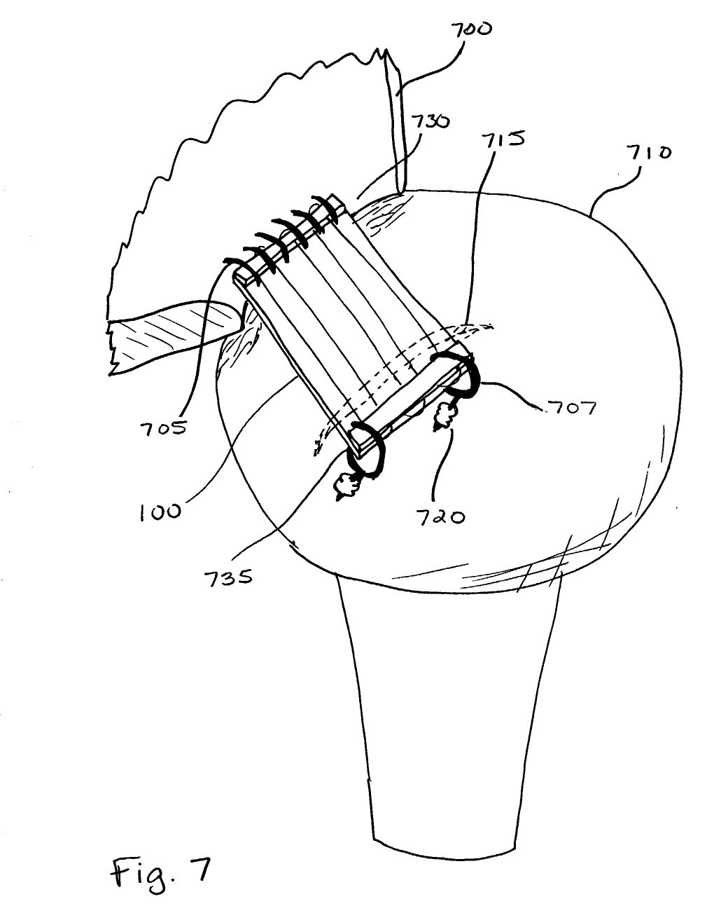

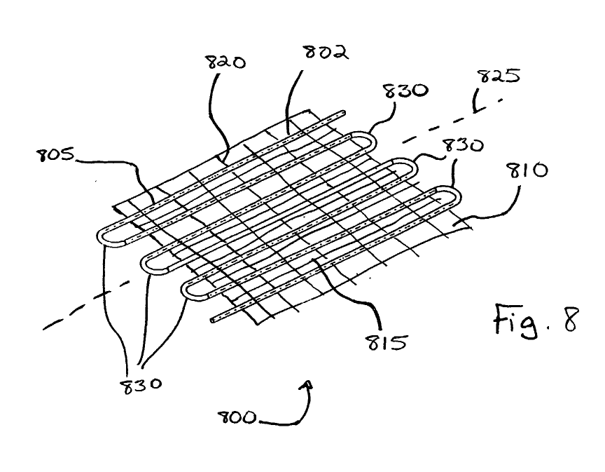

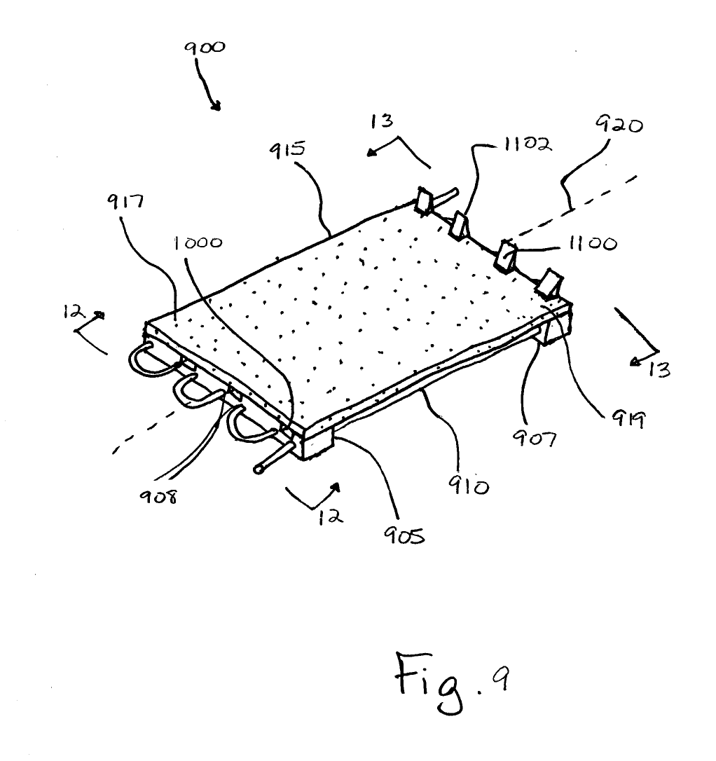

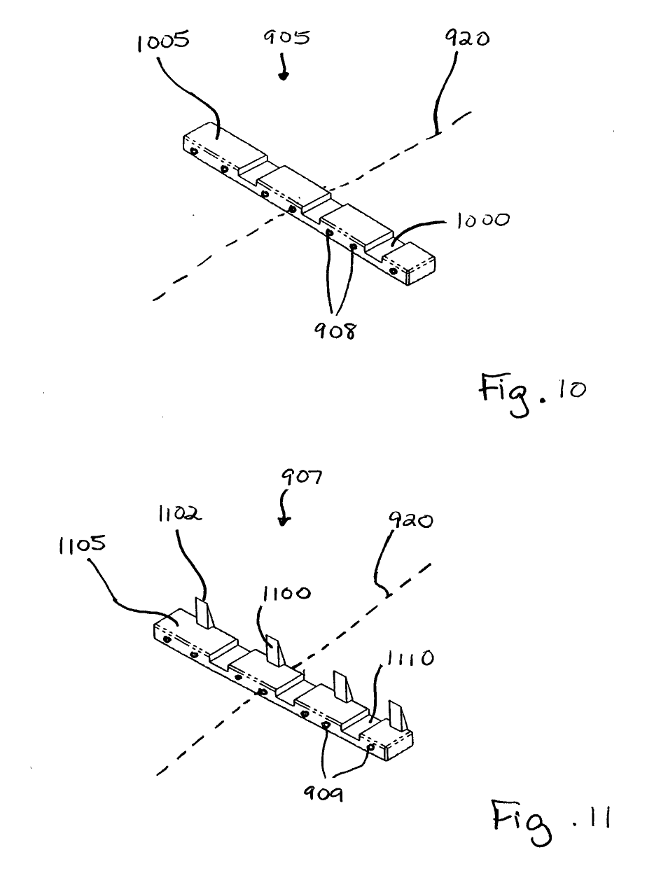

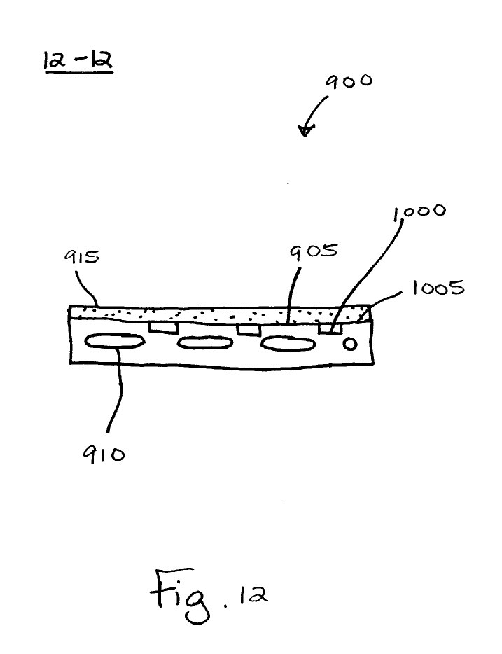

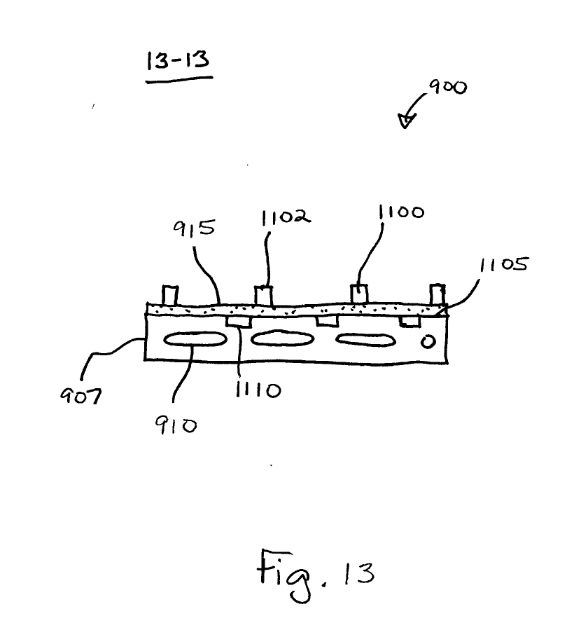

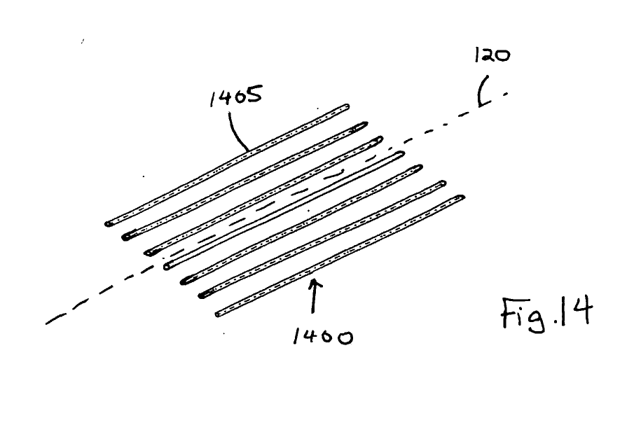

Referring to FIGS. 1-6, an implant 100 for tissue repair and implantation into tissue defects includes a planar substrate 115, two, spaced support members 105 attached to the substrate 115, and a flexible member 110. The flexible member 110 loops back and forth between support members 105 forming elongate elements 112 oriented along a longitudinal axis 120 of the implant 100. The elongate elements 112 form elbows 114 in regions beyond the substrate 115. The substrate 115 has first and second end regions 117, 119. Each of the support members 105 is attached to one of the end regions 117, 119, for example, by melting or welding the members 105 to the regions 117, 119. The support members 105 define through holes 107 through which the flexible member 110 is threaded.

The coupling of the flexible member 110 to the support members 105 at distinct regions defined by the through holes 107 acts to maintain the spacing, L, between the elongate elements 112. This design maintains the structural integrity of the implant 100 and transfers or distributes the load from the tissue evenly throughout the flexible member 110, and in particular, to all of the elongate elements 112 when the implant 100 is attached to tissue. Additionally, the support members 105 provide a tissue attachment site, as discussed below.

Referring particularly to FIG. 5, each of the support members 105 has a rectangular shape for receiving the flexible member 110. The support member 105 has sides 150 that are perpendicular to the longitudinal axis 120, with each side 150 having a height 155, for example, about 0.045″, and a width 160, for example, about 0.1″, sufficient to accommodate at least a portion of the flexible member 110.

The material used to form the support members 105 is injection molded around the flexible member 110, thus defining the holes 107 in the support members 105 that receive the flexible member 110. The molding of the material used for the support members 105 begins with preparation of a mold that corresponds to the shape of the support members 105. The flexible member 110 is placed within the mold, which is then sealed. The material used to form the support members 105 is heated and subsequently injected into the mold to encompass the flexible member 110. After cooling, the mold is removed and the support members 105 are ready to be attached to the substrate 115.

The substrate 115 is made of a material, such as non-woven felt, that is capable of being seeded with and supporting the attachment and proliferation of cells (seeding efficiency is discussed in detail below). The substrate 115 spans between support members 105 to overlay a substantial portion of the flexible member 110. This has the advantage that cells, once seeded onto the substrate 115, may proliferate evenly throughout the support members 105 and/or the flexible member 110 to generate tissue growth and/or facilitate tissue repair.

Materials used for the substrate 115 include solid materials; bioresorbable materials, that is, those materials that breakdown at a rate that allows the implant 100 to maintain sufficient integrity while the tissue is repairing and to encourage tissue repair; non-woven materials; and non-bioresorbable materials, that is, materials that retain their initial mechanical properties and maintain their strength after implantation.

Bioresorbable materials include polymers or copolymers such as hydroxy acids, particularly lactic acid and glycolic acid; caprolactone; hydroxybutyrate; dioxanone; orthoesters; orthocarbonates; aminocarbonates; and trimethylene carbonate.

Appropriate non-bioresorbable materials include polyesters, particularly aromatic polyesters, such as polyalkylene terephthalates, like polyethylene terephthalate and polybutylene terephthalates; polyamides; polyalkenes such as polyethylene and polypropylene; poly(vinyl fluoride), polytetrafluoroethylene carbon fibers, silk (natural or synthetic), carbon fiber, glass, and mixtures of these materials. Solid materials include woven or non-woven fibrous or fleece material; foam; sponge; dendritic material; knitted, braided, or crocheted material; or a mixture of two or more of these. Non-woven materials include fibers that are either dry laid, wet laid, spun laid, or melt blown. The fibers of the non-woven material can be arranged to give a random entanglement providing a large surface area for cell attachment or capture during cellular in-growth. The void fraction (that is, the fraction of the volume of void relative to the volume of the non-woven material) of the non-woven material is in the range 50 to 99%, and is preferably in the range 90 to 99%.

In general, materials used for the flexible member 110 are fabrics that are woven, knitted, braided, crocheted, straight, twisted, or a mixture of these. The fabric can be modified to give a specific architecture, allowing, in their turn, properties like percentage open volume, toughness, and other characteristics to be accurately tailored to the specific application. In one implementation, the elongate elements 112 are made of braided material, which has a favorable load to elongation relationship, strength, and elasticity. The braided material includes interwoven yarns, with each yarn including a group of fibers. The braided material can be 120 denier fiber (that is, 64 fibers) having a 6-7 ply core, with 16 end braid and an outer core having a diameter in the range of 0.5-0.7 mm. In another implementation, the elongate elements 112 are made of No. 2 suture that is either resorbable or non-resorbable.

Generally, materials used for the support members 105 and the flexible member 110 include bioresorbable materials and non-bioresorbable materials. Appropriate bioresorbable materials include polymers or copolymers such as hydroxy acids, (particularly lactic acid, and glycolic acid), caprolactone, hydroxybutyrate, dioxanone, orthoesters, orthocarbonates, aminocarbonates, trimethylene carbonate; natural materials such as, collagen, cellulose, fibrin, hyaluronic acid, fibronectin, chitosan, or mixtures of two or more of these materials. Appropriate non-bioresorbable materials include polyesters, particularly aromatic polyesters, such as polyalkylene terephthalates, like polyethylene terephthalate and polybutylene terephthalates; polyamides; polyalkenes such as polyethylene and polypropylene; poly(vinyl fluoride), polytetrafluoroethylene carbon fibers, silk (natural or synthetic), carbon fiber, glass, and mixtures of these materials.

The seeding efficiency of the substrate 115 is greater than 50% and preferably greater than 70%. Seeding efficiency is determined in the following way: a 2 mm×10 mm disc including the substrate is soaked overnight in fetal calf serum. The disc is then seeded with four million HuFF (human foreskin fibroblast) cells, suspended in 1 ml of culture medium by pulling the cell suspension backwards and forwards through the disc five times, using a 1 ml pipette. The proportion of cells adhering to the disc is determined (by DNA assay using Hoechst 33258T dye) after which the seeding efficiency is calculated as the percentage of cells adhering to the disc in relation to the total number of cells.

In one preferable implementation, the support members 105 and the flexible member 110 are made of poly(lactic acid) (PLA) and the substrate 115 is made of poly(glycolic acid) (PGA).

The implant 100 can include cells that are incorporated, for example, into the substrate 115, either before or after implantation into the tissue. If carried out before implantation, tissue growth may be carried out exclusively in vivo but can also be preceded by in vitro tissue culturing.

The cells are normally incorporated into the implant 100 by means of a carrier medium, that is, a medium that is no longer present in the implant after the cells have been seeded and remain embedded in the implant. Examples of this type of carrier medium are cell culture media and hydrogel. Preferably, a cell culture medium is employed to seed the cells, for example DMEM (DULBECO'S™ Modified Eagle's Medium containing 10% calcium).

If the carrier medium is a hydrogel it is incorporated within and/or on and/or around at least the substrate 115. Preferably, the carrier medium is incorporated at least within the substrate, since this efficiently utilizes the available open volume for cellular growth. More preferably, the carrier medium occupies the entire open volume of the substrate. Hydrogels that may be used as carrier media include positively charged, negatively charged, and neutral hydrogels that may be saturated or unsaturated. Examples of hydrogels are TETRONICS™ and POLOXAMINES™, which are poly(oxyethylene)-poly(oxypropylene) block copolymers of ethylene diamine; polysaccharides, chitosan, poly(vinyl amines), poly(vinyl pyridine), poly(vinyl imidazole), polyethylenimine, poly-L-lysine, growth factor binding or cell adhesion molecule binding derivatives, derivatised versions of the above (for example, polyanions, polycations, peptides, polysaccharides, lipids, nucleic acids or blends, block-copolymers or combinations of the above or copolymers of the corresponding monomers); agarose, methylcellulose, hydroxyproylmethylcellulose, xyloglucan, acetan, carrageenan, xanthan gum/locust beangum, gelatin, collagen (particularly Type 1), PLURONICS™, POLOXAMERS™, POLY(N-isopropylacrylmide) and N-isopropylacrylmide copolymers.

The cells with which the implant 100 can be seeded include cells that are terminally differentiated or capable of undergoing phenotypic change, for example, stem cells, pluripotent cells, and other precursor cells. More specifically, mesenchymal, tenocytes, ligamentous, and chondrocytic cells may be seeded into the synthetic implants.

As stated above, it is preferred to seed the implant 100 and to at least partially grow tissue within the implant in vitro prior to implantation of the implant. With this in mind, the implant can include biological tissue that is formed from the partially grown tissue within the implant. In the event that the implant is seeded with cells and biological tissue is grown in vitro, then, if the support member, the flexible member, or the substrate includes a resorbable material, then that support member, flexible member, or substrate, as the case may be, can be resorbed prior to implantation. Preferably, the substrate is resorbable and is substantially resorbed prior to implantation.

Accordingly, the implant 100 in which tissue has partially grown includes a support member, a flexible member, and a biological tissue that is generated by culturing the cell-seeded implant such that the substrate has substantially resorbed. As employed here, the term “culturing” means supplying with nutrients and maintaining conditions (for example, temperature, pH) that propagate tissue growth.

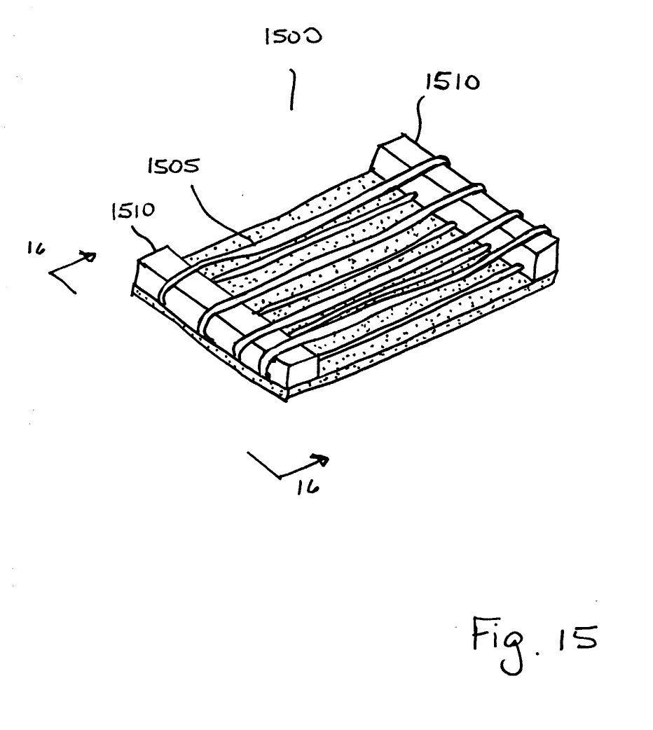

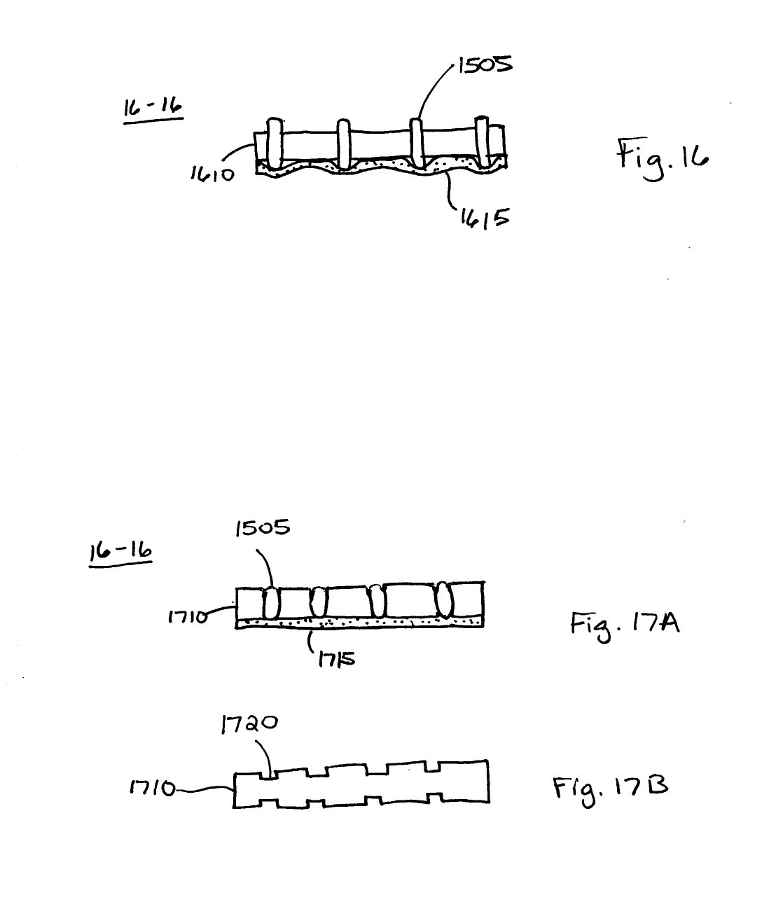

Referring also to FIG. 7, in use, the implant 100 is attached to tissue 700 to be repaired. For example, the tissue 700 has been detached from bone 710 at an original tissue/bone interface 715 and the implant 100 is used to repair this tear by attaching the tissue 700 to the bone 710 and providing a matrix within which tissue is grown. In particular, a first end 730 of the implant 100 is attached to the tissue 700 by attaching suture 705 to the support member 105 at the first end 730 and attaching the suture 705 to the tissue 700. The suture 705 is attached to the support member 105 by looping and stitching the suture 705 between elements 112. The suture 705 is attached to the tissue 700 using a stitching technique. Also, a second end 735 of the implant is attached to the bone 710 at or near the interface 715 by attaching suture 707 to the support member 105 at the second end 735 and attaching the suture 707 to the bone 710 using suture anchors 720. The suture 707 is attached to the support member 105 by looping and stitching the suture 707 between elements 112. The suture 707 is attached to the bone 710 using suture anchors 720 that are designed with sharp ends 725 to enter and grasp the bone 710.

In general, the implant 100 can be implanted into a wound site in a mammalian organism in clinical need. The term “wound site” means any site where a change in the tissue from its normal condition has occurred, for example, a change as a result of traumatic insult or degenerative changes. For example, as shown in FIG. 7, at the wound site, the tissue 700 has been torn from the bone 710 at the interface 715.

After attachment, the implant 100 provides a matrix within which tissue grows. The tissue that is grown in the matrix strengthens the implant 100 and bridges the gap between the torn tissue 700 and the bone 710. Any bioresorbable material within the implant 100 will breakdown as the tissue 700 and bone 710 repairs.

The implant 100 can be used for the partial or total replacement of tissue, particularly connective tissue such as ligament, cartilage, bone, meniscus, tendon, skin and the like. For example, in FIG. 7, the tissue 700 torn from the bone 710 is a tendon from a rotator cuff. Ligaments that can be totally or partially replaced include the medial and lateral collateral ligaments, the anterior and posterior cruciate ligaments (ACL and PCL respectively) and ligaments and tendons of the elbow and hand. Tendons that can be totally or partially replaced include the Achilles tendon and patellar tendon. The implants are particularly effective in the total or partial replacement of the rotator cuff of the glenohumeral joint.

The rotator cuff is made of four tendons, that is, the supraspinatus, infraspinatus, teres minor, and subcapularis. Ruptures to the supraspinatus are the most common problem encountered. For rotator cuff applications, the implant can be shaped in a generally triangular configuration, as shown in EP 0 7 44 165 or in the short or long Y shape as utilized 5 in the RCR™ device commercially available from Merck Biomaterial, France. Alternatively, if reinforcement is a goal, the implant can be shaped like a strip, that is, a shape having a length that is much greater than a width. Rotator cuff implants are secured in place by any conventional technique, for example, suturing, suture anchors, bone fixation devices, and bone screws. As shown in FIG. 7, as an example, the implant 100 is secured in place using sutures 705, 707 and suture anchors 720.

The implant 100 can be used to partially or totally replace or augment other tissues such as the ACL, medial collateral ligament (MCL), posterior cruciate ligament (PCL), patella tendon, lateral collateral ligament (LCL) and ligaments and tendons of the elbow and hand. The implant can be used to repair a patellar tendon harvest site. Typically, when the patellar tendon is harvested from a patient, a portion of the tendon (for example, the middle one third of the tendon) is harvested with patella and tibia bone plugs integrally attached thereto. The use of the harvested patellar tendon for reconstruction of ligaments is considered advantageous because the tissue is derived from the host patient and the implanted tendon readily allows rapid tissue ingrowth.

To repair the patellar tendon harvest site, the implant 100 is implanted into the site following harvesting of the bone-patellar-tendon-bone graft and secured, for example, by fixing the implant into the site. The implant can be disposed along the length of the patellar tendon and secured to the remaining natural patellar tendon, for example, by suturing opposite sides of the implant to the tendon. The implant can be secured to the tibia and patella, for example, by cementing, suturing, stapling, or fixing with one or more screws.

The implant 100 can be used to repair a ruptured or torn Achilles tendon, for example, tears that occur within the Achilles tendon itself, severing the tendon, or ruptures, which result from the tendon tearing off of the calcaneus. For Achilles tendon repair, the implant is made of elements that have load-bearing properties similar to the naturally occurring Achilles tendon and is designed so as to allow new Achilles tendon growth on the implant. For Achilles tendon repair, the implant preferably includes a bioresorbable material that has the property of resorbing slowly in the body, such as, for example, PLA. The slow resorption allows retention of the mechanical properties of the implantable material until a time when the newly reconstructed Achilles tendon can take over the load-bearing functions of the implant.

To repair a tom or ruptured Achilles tendon, standard surgical methods of identifying and locating the torn tendon are used. Briefly, a longitudinal incision is made just medial to the Achilles tendon and the severed end(s) of the ruptured tendon identified. Where the Achilles tendon is severed from within, the opposite ends of the implant are attached to each of the torn tendon ends, for example, by suturing the first end of the implant to the first end of the torn Achilles tendon and suturing the second end of the implant to the second end of the torn Achilles tendon, thereby reattaching the first and second ends. Alternatively, where the Achilles tendon is torn away from the calcaneus, the surgical method includes attaching a first end of the implant to the calcaneus and the second end of the implant to the torn end of the Achilles tendon, for example, by suturing, thereby reattaching the Achilles tendon to the calcaneus.

Other implementations are within the scope of the following claims.

For example, in another implementation, before attachment, the implant 100 is cut to reduce a width (perpendicular to the axis 120) of the implant to a width suitable for the tissue repair location. Alternatively, the implant is presented in a modified form suitable for different surgical applications. For example, two or more implants can be superimposed, mutually connected, and also cut, if appropriate. Suitable methods for attaching the superimposed implants to one another include, for example, stitching, crocheting, impregnation with a binder, an adhesive, or heat sealing.

In a further implementation, two or more implants 100 can be joined in a serpentine or “concertina”-type of arrangement. One or more implants can be placed on top of each other, aligned along their respective longitudinal axes and then rolled, attached parallel to one another, plaited together, or twisted together. The implants can be rolled or wound in a shape and then joined with other rolled implants to form a tube, a form referred to as “Swiss Roll” form. For example, implants in the form of a “Swiss Roll” can have a diameter in the range 5-15 mm. In this design, the windings can be interconnected by stitching or crocheting, by impregnation with a binder, by use of an adhesive or by heat sealing to prevent unraveling.

Referring also to FIG. 8, in another implementation, rather than flexible member 110, the implant includes a flexible member 800 shaped as an elongate fabric tape. The flexible member 800 is made of a strand 802 attached to a mesh 810. The mesh 810 includes warp strands 815 (that is, strands that are parallel with a longitudinal axis 825) and weft strands 820 (that is, strands that are perpendicular with the longitudinal axis 825) that provide additional structural integrity to the flexible member 800. The flexible member 800 can be heat sealed or sealed with a binder in order to prevent fraying at the edges of the elongate fabric tape. The strand 802 forms elongate elements 805 oriented along the longitudinal axis 825 of the implant to form elbows 830 in regions beyond the substrate. The elongate elements 805 are maintained in a spaced apart relationship by the mesh 810.

Referring to FIG. 9, in another implementation, an implant 900 for tissue repair includes a planar substrate 915 (substantially similar to substrate 115), two, spaced support members 905, 907, and a flexible member 910 (substantially similar to member 800 or 110). The substrate 915 has first and second end regions 917, 919 to which each of the support members 905, 907 are attached by, for example, melting or welding the members 105 to the regions 117, 119. The support members 905, 907 define through holes 908, 909 through which the flexible member 910 is threaded.

As shown in FIGS. 10 and 12, the support member 905 is formed with one or more troughs 1000 formed on a surface 1005 of the support member. As shown, the troughs 1000 are formed on a surface of the support member that faces the substrate 915, though the troughs 1000 can be formed on any suitable surface of the support member. The troughs facilitate bending and preserve flexibility of the support member 905 and the implant.

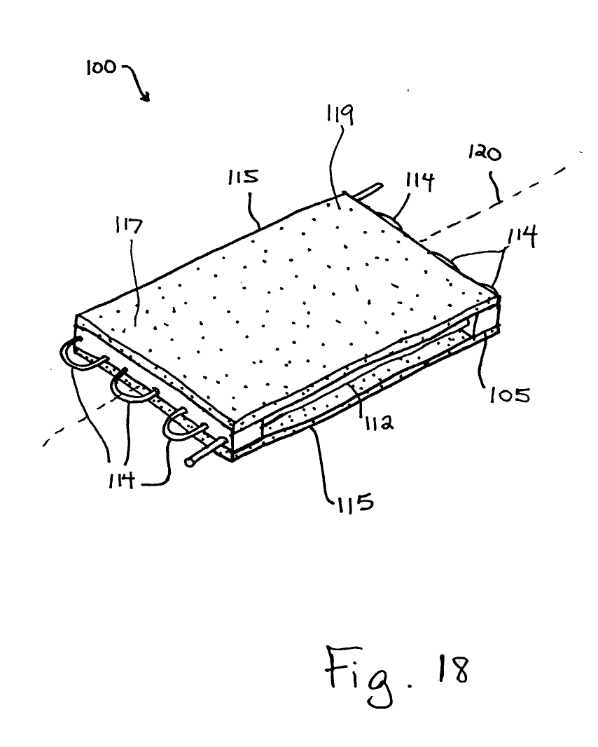

As shown in FIGS. 11 and 13, the support member 907 is formed with one or more spikes 1100 formed on a surface 1105 of the support member. As shown, the spikes are formed on a surface of the support member that faces the substrate 115, though the spikes can be placed on any suitable surface of the support member. The spikes are positioned such that, when the substrate 915 is coupled to the support member 907, the spikes pierce the substrate. Alternatively, the substrate may have a size small enough such that the spikes do not pierce the substrate when the substrate is coupled to the support member 907. The pointed ends 1102 of the spikes facilitate attachment of the implant 900 to the tissue. Thus, the spikes are shaped to have a sharp tip or a triangular cross section. The support member 907 is shown having troughs 1110, though the support member 907 can be formed without troughs. Additionally, the support member 905 is shown without spikes, though the support member 905 can be formed with spikes.

The implant can include any number of support members depending on the size of the implant and the level of support needed. For example, the implant can include a single support member positioned in a middle portion of the implant. The support members can be positioned at any suitable location of the implant as long as the support members maintain the structural integrity of the flexible member. For example, the support member can be positioned near a middle of the implant.

Although the substrate is shown as being rectangular, other geometric forms are possible. As shown in FIG. 14, the flexible member 1400 can be made of discrete elongate elements 1405 that are not attached at elbows.

The troughs 1000 can be shaped into any suitable form, such as, for example, steps (as shown), rounded and continuous with the surface 1005, or triangular.

A portion of the flexible member can be incorporated within the substrate or within both the substrate and the support member. In another implementation, the flexible member is threaded through the support members using a technique such as stitching, crocheting, by means of a binder, an adhesive, or by heat sealing. Generally, the flexible member also can be attached to the substrate. The flexibility of a support member depends on the material used in forming the support member and on the size, that is, the thickness, of the support member. For example, the support member becomes more rigid when the thickness is increased.

Referring also to FIG. 15, in another implementation of an implant 1500, a flexible member 1505 can be wrapped around support members 1510. As shown in FIG. 16, in one implementation, the flexible member 1505 is passed around support members 1610 that are formed like support members 105. Thus, a substrate 1615 is attached to the support members 1610 at those locations not covered by the flexible member 1505. As shown in FIGS. 17A and 17B, in another implementation, the flexible member 1505 is passed around troughs 1720 formed in support members 1710 to receive the flexible member 1505. The substrate 1715 is attached to the support members 1710 at locations not covered by the flexible member 1505 and lies flush with the support members 1710.

Referring also to FIG. 18, the substrate 115 can be positioned on both sides of the support members 105 and flexible member 110.

The support member and the flexible member also can be capable of being seeded with and supporting the growth of cells. In this event, the substrate has a higher seeding efficiency than the support member and the flexible member, according to the above definition of seeding efficiency.

The seeding efficiency of the substrate can be an inherent property of the material selected or the result of an addition treatment step. Examples of treatment steps that can be employed to achieve the requisite seeding efficiency include surface-modification by application of a material, such as serum, fibronectin or RGD peptide; a chemical method, such as acid hydrolysis; or plasma treatment.

The substrate is attached to at least a portion of the flexible member if cellular integration in a localized region of tissue is important. In another implementation, the flexible member can be encased in the substrate. In another implementation, the substrate is attached to at least a portion of the support member. Appropriate methods for attaching the substrate to the support member or the flexible member are stitching, crocheting, by means of a binder, an adhesive, or by heat sealing.

The implant can be formed in a shape other than the rectangular shape of the implant 100 described above. For example, the implant can be formed in a circular shape, in which case the substrate would have a circular shape and the implant would include a single support member that spanned a circumference of the substrate. The flexible member would loop back and forth along diagonals through the support member forming elongate elements that form elbows in regions beyond the substrate. In this way, the coupling of the flexible member to the support member at distinct regions defined by through holes in the support member acts to maintain the spacing between each of the elongate elements.

What is claimed is:

1. An implant for tissue repair comprising:
a support member;
a flexible member coupled to the support member; and
a substrate coupled to the support member and comprising a material capable of being seeded with and supporting the proliferation of cells;
wherein the support member is capable of distributing a load throughout the flexible member.

2. The implant of claim 1 wherein the substrate is attached to the support member.

3. The implant of claim 1 wherein the substrate is planar in form.

4. The implant of claim 1 wherein at least a portion of the flexible member is threaded into the support member.

5. The implant of claim 1 wherein at least a portion of the flexible member is molded into the support member.

6. The implant of claim 1 wherein the flexible member is wrapped around the support member.

7. The implant of claim 6 wherein the support member includes troughs formed to receive a portion of the flexible member that contacts the support member.

8. The implant of claim 1 wherein the flexible member comprises parallel elongate elements.

9. The implant of claim 8 wherein the support member serves to maintain a spacing between the parallel elongate elements.

10. The implant of claim 8 wherein the parallel elongate elements comprise a braided material.

11. The implant of claim 1 wherein the support member comprises a trough formed on a surface of the support member that impinges the substrate.

12. The implant of claim 1 wherein the support member comprises a spike configured to facilitate attachment of the implant to tissue.

13. The implant of claim 1 wherein the flexible member comprises one or more of a woven, knitted, braided, crocheted, straight, or twisted material, and a mixture of these.

14. The implant of claim 1 wherein the flexible member comprises poly(lactic acid).

15. The implant of claim 1 wherein the support member comprises one or more of a woven, non-woven, knitted, braided, crocheted material, and a mixture of these.

16. The implant of claim 1 wherein the support member comprises poly(lactic acid).

17. The implant of claim 1 wherein the substrate comprises one or more of a woven or non-woven material; a foam; a sponge; a dendritic material; a knitted, braided, a crocheted material; and a mixture of these.

18. The implant of claim 1 wherein the substrate comprises poly(glycolic acid).

19. The implant of claim 1 wherein one or more of the support member, the flexible member, and the substrate comprises bioresorbable or non-bioresorbable materials.

20. The implant of claim 19 wherein the bioresorbable material comprise bioresorbable polymers or copolymers comprising hydroxy acids, glycolic acid; caprolactone; hydroxybutyrate; dioxanone; orthoesters; orthocarbonates; or aminocarbonates.

21. The implant of claim 19 wherein the bioresorable material comprises poly(lactic) acid, poly(glycolic) acid, or a mixture of these.

22. The implant of claim 19 wherein the non-bioresorbable material comprises polyesters; polyamides; polyalkenes; poly(vinyl fluoride); polytetrafluoroethylene; carbon fibers; natural or synthetic silk; and mixtures of these materials.

23. The implant of claim 19 wherein the non-bioresorbable material comprises a polyester selected from polyethylene terephthalate and polybutylene terephthalate.

24. The implant of claim 1 further comprising cells seeded in the substrate.

25. The implant of claim 24 wherein the substrate includes a carrier medium for holding the cells.

26. The implant of claim 24 wherein the implant includes biological tissue grown from the cells.

27. A method of making an implant, the method comprising:
coupling a flexible member to a support member to form a flexible structure such that the support member is capable of distributing a load throughout the flexible member;
coupling a substrate to the flexible structure, the substrate including a material capable of being seeded with and supporting the proliferation of cells;
incorporating cells into the substrate; and
growing biological tissue within at least a portion of the substrate.

29. The method of claim 27 further comprising cutting the implant into a shape suitable for the selected site.

30. A method of treating a tissue harvest site, the method comprising:
providing an implant comprising:
a support member,
a flexible member coupled to the support member, and
a substrate coupled to the support member and comprising a material capable of being seeded with and supporting the proliferation of cells; and
implanting the implant at the harvest site.

31. The method of claim 31 wherein the tissue harvest site includes a rotator cuff.

32. The method of claim 31 wherein the tissue harvest site includes an anterior cruciate ligament.

33. The method of claim 31 wherein the tissue harvest site includes a posterior cruciate ligament.

34. The method of claim 31 wherein the tissue harvest site includes an Achilles tendon.

35. The method of claim 31 wherein the tissue harvest site includes a patellar tendon.

36. The method of claim 31 wherein the tissue harvest site includes a medial collateral ligament.

37. The method of claim 31 wherein the tissue harvest site includes a lateral collateral ligament.

38. The method of claim 31 wherein the tissue harvest site includes a ligament in the hand.

39. The method of claim 31 wherein the tissue harvest site includes a tendon in the hand.

40. The method of claim 31 wherein the tissue harvest site includes a ligament in the elbow.

41. The method of claim 31 wherein the tissue harvest site includes a tendon in the elbow.
